(19) United States
(12) Patent Application Publication
(10) Pub. No.: US 2023/0330343 A1
(43) Pub. Date: Oct. 19, 2023

(54) SAFETY SYRINGE WITH FLEXIBLE RESERVOIR AND METHOD OF USE

(71) Applicant: DONN R. DRESSELHUYS, Naples, FL (US)

(72) Inventor: DONN R. DRESSELHUYS, Naples, FL (US)

(21) Appl. No.: 18/103,150

(22) Filed: Jan. 30, 2023

Related U.S. Application Data
(63) Continuation-in-part of application No. 17/719,418, filed on Apr. 13, 2022.

(51) Int. Cl. A61M 5/28; A61M 5/32

(52) U.S. Cl. CPC A61M 5/282; A61M 5/3232

(57) ABSTRACT

A syringe assembly (1) having a flexible reservoir (2) containing fluid (3) at a proximal end, which is connected to a needle holding section (5) by a passageway (4) at an exit end of the reservoir. The reservoir has a seal (9) over the exit end to protect the fluid from exposure to air or contamination. The needle-holding section is threadably connected to the passageway so when the needle holding section is rotated inward toward the reservoir an inner portion of the needle (10) can pierce the seal to allow the fluid in the reservoir to pass through the needle into a user’s body when the reservoir is squeezed or depressed. A spring (12) attached to the needle holding section surrounds the length of the needle acts as a cover to help prevent accidental needle pricks.

### CROSS REFERENCE TO RELATED APPLICATIONS

This application is a continuation-in-part and thus claims the priority benefit of Patent Application No. 17/719,418 filed on Apr. 13, 2022.

### BACKGROUND OF THE INVENTION

This invention relates to syringes, more particularly a plunger-less syringe with pre-filled flexible fluid reservoir and a needle-prick prevention mechanism.

Conventional syringes primarily consist of a cylindrical reservoir for holding an injectable solution, a plunger at a proximal end and a needle at a distal end. In order to inject a medication or fluid solution using such a conventional syringe, one must first fill the reservoir with solution from a vial by inserting the needle into the vial with the plunger in an inward depressed position thereby creating a vacuum within the reservoir and then moving the plunger toward an outward extended position to allow the solution to flow into the reservoir. Once the desired solution is in the reservoir to inject it into the body one must insert the needle into the body and depress the plunger.

The above process of using a conventional syringe can be difficult for a person to accomplish by her or himself, especially if elderly or with infirmities, such as arthritis and thus, often requires the assistance of another person. If a second person is involved there is a risk that a second person can receive a needle stick which can be harmful or ultimately deadly if the receiver has a disease, such as aids.

A related problem with the common necessity of assistance from a second person is that a second person if even available is embarrassed or unwilling to assist.

A further problem with the use of conventional syringes is that such are usually not pre-filled and must be filled from a vial which may result in a patient receiving an inadequate or oppositely, an excessive dose of medication.

Another problem with conventional syringes is that the plungers and cylinders may become contaminated through age degradation, thereby causing a loss of vacuum, such that it does not function properly.

Even a further problem with conventional syringes results from dead space between the syringe hub and the needle in which fluid cannot be expelled from the syringe. The problems associated with dead space in syringes include medication waste and inadequate dosing.

Another problem with conventional syringes is reusability by secondary users, such as by individuals addicted to drugs, which can spread diseases, such as hepatitis and AIDS.

Finally, an even more serious problem with most conventional syringes is the inadvertent or accidental pricking of health care workers and others which can result in the transmission of diseases, such as AIDS. Needle pricking is a common occurrence which causes significant liability exposure and cost to health care facilities.

Thus, a need exists for a syringe that eliminates the above problems.

### SUMMARY OF THE INVENTION

The primary object of the present invention is to provide a syringe that enables a user to inject oneself without the assistance of another person.

Another object of the present invention is to provide such a syringe that is prefilled with the proper dosage of medication or other solution for single dose, single usage.

A further object of the present invention is to provide such a syringe that is safe to use by avoiding a likelihood of needle sticks to others.

An additional object of the present invention is to provide a syringe that is for one-time use, thereby eliminating reuse by drug users, thereby eliminating the possible spread of diseases.

The present invention fulfills the above and other objects by providing a syringe having a flexible reservoir at a proximal end threadably connected by a passageway to a needle holding section at a distal end. The reservoir can be prefilled with an injectable medication or other solution and hermetically sealed to avoid exposure to air and contamination. The needle holding section contains a needle having an inner proximal portion which extends into the passageway and an outer portion at a distal end for injecting fluid into a user’s body. The needle may be covered by a slide on cap until used.

Alternatively, or in addition to a cap, the syringe may employ a spring surrounding the needle which depresses inward when pressed against a receiving person’s skin during injection which recovers to its extended position after injection so as to keep the needle from accidentally pricking a health care worker or other person.

A method of use involves rotating the rotatable needle holder inward until the inner end of the needle pierces the hermetically sealed reservoir. Then the needle with spring cover can be depressed against the receiving person’s skin with enough pressure to depress the spring needle cover so that the needle is inserted into the skin at which time the flexible reservoir can be squeezed between fingers or depressed by a thumb to push the injectable fluid through the needle in the passageway and out the needle into the receiving person. Then, once injection has been completed, the needle is removed outward from the receiving person as the spring needle cover extends to its original position surrounding the needle, thereby preventing accidental needle pricks.

The special design of the syringe of the present invention results in automatic disablement of the syringe so it cannot be reused for two reasons; one, the reservoir has been pierced during use by the inner end of the needle, meaning the reservoir cannot be refilled; and two, after use the reservoir is collapsed so there is no space for fluid.

The above and other objects, features and advantages of the present invention should become even more readily apparent to those skilled in the art upon a reading of the following detailed description in conjunction with the drawings wherein there is shown and described illustrative embodiments of the invention.

### DESCRIPTION OF THE DRAWINGS

In the following detailed description, reference will be made to the attached drawings in which:

FIG. 1 is a side view of the entire syringe assembly of the present invention;

FIG. 2 is an exploded side view illustrating the components of the syringe assembly of the present invention;

FIG. 3 is a side plan view illustrating the internal features of the syringe assembly of the present invention;

FIG. 4 is a side view of the syringe with a needle spring cover in a rest position prior to injection;

FIG. 5 is a side partial cutaway view of the syringe with a needle spring cover in a depressed position during injection; and

FIG. 6 is a side view of the syringe with a needle spring cover in an extended position after injection.

### DESCRIPTION OF THE PREFERRED EMBODIMENTS

For purposes of describing the preferred embodiment, the terminology used in reference to the numbered components in the drawings is as follows:
1. syringe, generally
2. flexible reservoir
3. fluid in reservoir
4. passageway
5. needle holding section
6. distal needle portion
7. needle cover
8. threads on passageway
9. reservoir seal
10. proximal needle portion
11. threads in needle holder
12. needle spring cover
13. receiving person

Referring to the drawing figures, FIG. 1 illustrates a side view of the syringe assembly 1 having a flexible reservoir 2 in which medication or other fluid 3 to be injected can be contained. The reservoir 2 is connected by a passageway 4 to a needle holding section 5 and a separate needle cover 7. Although the flexible reservoir 2 is illustrated as being in the shape of bulb, the reservoir 2 could be of almost any shape, oblong, tube-like or other shape just so long as it is flexible so it can be squeezed to expel fluid when squeezed between fingers or depressed by a thumb.

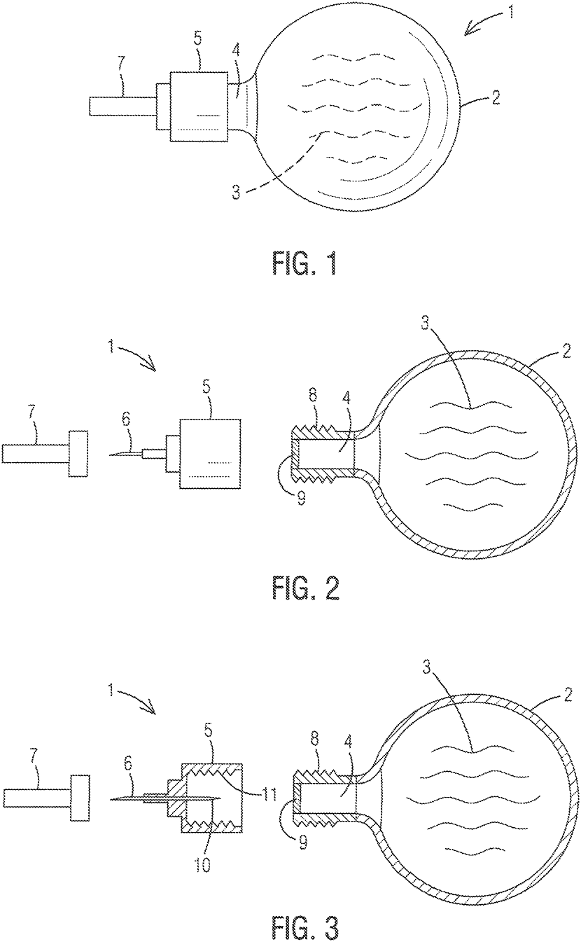

FIG. 2 illustrates an exploded side view of a syringe assembly 1 of the present invention showing the flexible reservoir 2 containing fluid 3 with passageway 4 having external threads 8 for rotatable connection to the internal threads 10 (shown in FIG. 3) in the needle holding section 5. The exit end of the passageway 4 preferably would have a seal 9 to hermetically seal and protect from exposure to air or contamination of the fluid 3 within the reservoir 2 until needed and ready for injection. The seal 9 would be pierceable by the internal proximal needle portion 10 as in the method of use described hereinafter.

FIG. 3 illustrates an exploded side plan view of the syringe assembly 1 of the present invention which best shows the internal features. As previously illustrated and described the syringe assembly 1 has a flexible reservoir 2 containing injectable fluid 3 hermetically sealed by seal 9 at the exit end of the passageway 4. The passageway 4 contains external threads 8 which are rotatably connectable to internal threads 11 in the needle holding section 5. The needle would have an internal proximal portion 10 which is designed to pierce the seal 9 in the passageway 4 when the needle holding section 5 is rotated inward sufficiently so as to allow the release of the fluid 3 in the reservoir 2 when squeezed or depressed. Remaining components of the syringe 1 shown in FIG. 3 include the external portion of the needle 6 and needle cover 7.

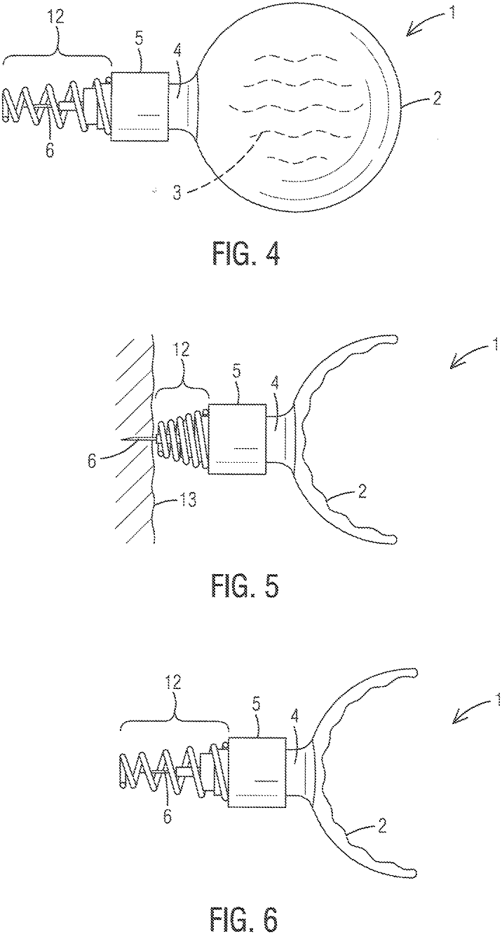

FIG. 4 illustrates the syringe assembly 1 of the present invention having a spring 12 surrounding the needle 6 to act as a cover for preventing inadvertent or accidental needle pricks. The spring 12 preferably is permanently attached or secured to a distal end of the needle holding section 5 by compression, indentation, epoxy or other securing means.

FIG. 5 illustrates the syringe assembly 1 during injection with the spring 12 in a compressed position to allow the needle 6 to penetrate the skin 13 of a receiving person 13. Although the spring 12 could take almost any configuration, it may preferably be pyramidal in shape as shown to allow the outer band of the spring to compress within the inner bands. The spring 12 would have a spring constant that keeps it from being easily depressed to prevent easy needle exposure, but at the same time allows injection without excessive pressure.

The final illustration in FIG. 6 shows the syringe assembly 1 after injection with the needle spring 12, once again in a fully extended position covering the needle 6, so as to prevent accidental needle pricks. Although the fluid reservoir 2 is flexible, due to removal of the medicinal fluid 3 from within it would likely be crushed as shown after injection.

A method of using the syringe 1 of the present invention involves obtaining it in a pre-filled status with the flexible reservoir 2 containing a desired amount of medication or other fluid 3. Then the user would rotate inward between two fingers the needle holding section 5 until the internal portion of the needle 10 pierces the hermetic seal 9, thereby allowing the fluid 3 within the reservoir 2 to flow into the needle 10 in the passageway 4. Next the user would remove the needle cover 7, or alternatively, if provided with a spring cover 12, press the distal end of the spring cover 12 against the skin of a receiving person to allow insertion of the external distal portion of the needle 6 into the body and finally, sq depressing with a thumb the flexible reservoir 2 until the fluid is emitted from the external distal portion of the needle 6 into the body to collapse the outer half of the reservoir 2 into the front half. To preclude inadvertent piercing of the seal 9 prior to use either or both thread sections 11 or 8 may have a stop such as a one slightly larger thread so that a slightly noticeable exertion during rotation of the needle section 5 is required to extend the internal portion of the needle 10 through the seal 9 and is thus rendered nonreusable.

The special design of the syringe of the present invention results in automatic disablement of the syringe so it cannot be reused for two reasons; one, the reservoir seal 9 has been pierced during use by the inner end of the needle 10, meaning the reservoir cannot be refilled; and two, after use the reservoir 2 is collapsed so there is no space for fluid, as is illustrated in FIG. 6.

The syringe assembly 1 preferably would be made of plastic and the reservoir being composed of flexible rubber or plastic with the needle of course being made of metal.

It is to be understood that while preferred embodiments of the invention have been described, it is not to be limited to the specific form or arrangement of parts herein described and shown. It will be apparent to those skilled in the art that various changes may be made without departing from the scope of the invention and the invention is not to be considered limited to what is shown and described in the specification and/or drawings.

I claim:

1. A syringe assembly comprising:
a flexible reservoir at a proximal end of the syringe assembly;
a needle-holding section at a distal end of the syringe assembly having an injection needle extending therefrom; and
a passageway at an exit end of the reservoir connecting the reservoir to the needle-holding section.

2. The syringe assembly of claim 1 wherein the reservoir is prefilled with fluid.

3. The syringe assembly of claim 1 wherein the passageway further comprises: a seal at the exit end of the reservoir to protect the fluid from exposure to air and contamination.

4. The syringe assembly of claim 1 wherein the needle-holding section is threadably connected to the passageway.

5. The syringe assembly of claim 4 wherein the needle in the needle-holding section has an interior proximal portion that extends into the exit passageway of the reservoir and an exterior distal portion for injecting the fluid into a user.

6. The syringe assembly of claim 5 wherein the proximal end of the needle of the is capable of piercing the seal when the needle-holding section is rotated inward toward the reservoir.

7. The syringe assembly of claim 5 further comprising:
a cover over the exit end of the needle.

8. The syringe assembly of claim 1 wherein the flexible reservoir is bulb shaped.

9. The syringe assembly of claim 1 further comprising:
a spring secured to the needle-holding section, said spring surrounding and
exceeding at least a length of the injection needle to act as a cover to prevent inadvertent needle pricks.

10. A syringe assembly comprising:
a flexible reservoir at a proximal end of the syringe assembly;
a needle-holding section at a distal end of the syringe assembly having an injection needle extending therefrom;
a passageway at an exit end of the reservoir connecting the reservoir to the needle-holding section;
wherein the reservoir is prefilled with fluid;
wherein the passageway further comprises a seal at the exit end of the reservoir to protect the fluid from exposure to air and contamination;
wherein the needle-holding section is threadably connected to the passageway of the reservoir;
wherein the needle in the needle-holding section has an interior proximal portion that extends into the exit passageway of the reservoir and an exterior distal portion for injecting the fluid into a user; and
a spring secured to the needle-holding section, said spring surrounding and exceeding at least a length of the injection needle to act as a cover to prevent inadvertent needle pricks.

11. A method of using the syringe assembly of claim 10 comprising the steps of: rotating the needle-holding section inward toward the flexible reservoir until the proximal portion of the needle pierces the seal;
pressing the spring against a skin of a receiving person, thereby depressing the spring and inserting the distal portion of the needle into a user’s body;
squeezing the flexible reservoir to cause the fluid in the reservoir to be expelled from the reservoir through the needle and into the user’s body; and
removing the needle from the user’s body while allowing the spring to extend around the needle to act as a cover to prevent accidental needle pricks.
